(19) United States
(12) Patent Application Publication
(10) Pub. No.: US 2023/0174924 A1
(43) Pub. Date: Jun. 8, 2023

(54) METHODS OF CULTURING PROBIOTIC BACILLI

(71) Applicants: The State of Israel, Ministry of Agriculture & Rural Development, Agricultural Research Organization, Rishon-LeZion (IL); Yissum Research Development Company of the Hebrew University of Jerusalem Ltd., Jerusalem (IL)

(72) Inventors: Moshe SHEMESH, ModiIn (IL); Shmuel GALILI, Petach-Tikva (IL); Ram REIFEN, Rehovot (IL)

(73) Assignees: The State of Israel, Ministry of Agriculture & Rural Development, Agricultural Research Organization, Rishon-LeZion (IL); Yissum Research Development Company of the Hebrew University of Jerusalem Ltd., Jerusalem (IL)

(21) Appl. No.: 18/098,184

(22) Filed: Jan. 18, 2023

Related U.S. Application Data
(63) Continuation of application No. PCT/IL21/50880, filed on Jul. 19, 2021.
(60) Provisional application No. 63/053,651, filed on Jul. 19, 2020.

(51) Int. Cl. C12N 1/20; C12P 17/12; A23L 5/46; A23L 13/40

(52) U.S. Cl. CPC C12N 1/20; C12P 17/12; A23L 5/46; A23L 13/40; C12R 2001/125

(57) ABSTRACT

A method of culturing bacteria of the Bacilli class is disclosed. The method comprises:
(a) adding the bacteria to a medium comprising pasteurized starch fibers of a legume of a leguminous plant, and
(b) culturing the bacteria under conditions that promote generation of a biofilm of the bacteria on the starch fibers.

### RELATED APPLICATIONS

This application is a Continuation of PCT Patent Application No. PCT/IL2021/050880 having International filing date of Jul. 19, 2021, which claims the benefit of priority under 35 USC § 119(e) of U.S. Provisional Patent Application No. 63/053,651 filed on Jul. 19, 2020. The contents of the above applications are all incorporated by reference as if fully set forth herein in their entirety.

### FIELD AND BACKGROUND OF THE INVENTION

The present invention, in some embodiments thereof, relates to a method of culturing Bacilli bacteria and use of the culture for generating food products and pigments.

Many bacterial species exist in natural settings as matrix-enclosed multicellular cells called biofilm. Bacillus subtilis is a beneficial Gram-positive, spore-forming bacterium ubiquitously found in soil, gastrointestinal tract (GIT) of ruminants/humans and also in food processing plants. B. subtilis inclines to form diverse biofilm phenotypes including pellicle or biofilm bundles in liquid media, and colony-type biofilm in solid media. Environmental and physiological conditions often dictate the B. subtilis cells for instigating the biofilm mode of growth.

Bacillus species belong to a versatile class of microbes that may provide numerous health benefits for the host organism. Some of those species are known to antagonize bacterial pathogens, while others protect or/and promote the growth of probiotic bacteria. In addition, Bacillus-based probiotics have shown to improve the digestive health by strengthening the intestinal barrier function and by attenuation of the inflammatory response in humans.

Probiotic Bacillus species have also a propensity to colonize human gut transiently. However, the prospect of probiotic survival in the acidic environment of human GIT is either infrequent or virtually nil. Several studies have been conducted previously to enhance a survival of probiotic species in GIT using food matrices [11-13].

Plant-based milks are gradually gaining attention due to their copious nutritional values. It is usually prepared by crushing legumes or nuts with 6 to 8 volumes of water. Lately, there is substantial interest in preparing chickpea (Cicer arietinum) based milks. Chickpea seeds are rich in healthy nutrients, minerals, proteins, carbohydrates and dietary fibers. It also has traditional standards and is a popular cuisine in India, middle-east and Mediterranean.

### SUMMARY OF THE INVENTION

According to an aspect of the present invention there is provided a method of culturing bacteria of the Bacilli class, the method comprising:
(a) adding the bacteria to a medium comprising pasteurized starch fibers of a legume of a leguminous plant, and
(b) culturing the bacteria under conditions that promote generation of a biofilm of the bacteria on the starch fibers, thereby culturing the bacteria.

According to an aspect of the present invention there is provided a method of generating a pigment comprising:

(a) culturing bacteria belonging to the genus Bacillus on a medium comprising pasteurized starch fibers of a legume of a leguminous plant under conditions that allow the secretion of the pigment into the medium; and

(b) collecting the medium.

According to an aspect of the present invention there is provided a culture comprising bacteria belonging to the class Bacilli and a medium comprising starch fibers of a pasteurized legume of a leguminous plant.

According to an aspect of the present invention there is provided a method of coloring a food product comprising combining a pigment generated by Bacillus subtilis with the food product under conditions that alter the color of the food product, thereby coloring the food product.

According to an aspect of the present invention there is provided a food product comprising a pigment generated by Bacillus subtilis, wherein the food is of a different color in the presence of the pigment as compared to in the absence of the pigment.

According to an aspect of the present invention there is provided a food product comprising the culture described herein.

According to an aspect of the present invention there is provided a culture medium comprising chickpea milk fortified with exogenous chickpea starch fibers.

According to embodiments of the invention, the medium is chickpea milk.

According to embodiments of the invention, the chickpea milk is fortified with exogenous chickpea fibers.

According to embodiments of the invention, the medium is a growth medium selected from the group consisting of lysogeny broth (LB), lysogeny broth enriched with glycerol and manganese (LBGM), milk and Man, Rogosa and Sharpe (MRS) medium.

According to embodiments of the present invention, the bacteria belong to the genus Bacillus.

According to embodiments of the present invention, the bacteria are comprised in a biofilm on the starch fibers.

According to embodiments of the present invention, the leguminous plant is selected from the group consisting of a plant of the genus Glycine, a plant of the genus Phaseolus, a plant of the genus Cicer, a plant of the genus Pisum, a plant of the genus Lens, a plant of the genus Cajanus, a plant of the genus Vicia, and a plant of the genus Arachis.

According to embodiments of the present invention, the leguminous plant selected from the group consisting of chickpea (Cicer arietinum), soybean (Glycine max), common bean (Phaseolus vulgaris), pea (Pisum sativum), lentil (Lens culinaris), pigeon pea (Cajanus cajan), broad bean (Vicia faba) and peanut (Arachis hypogaea).

According to embodiments of the present invention, the leguminous plant is chickpea.

According to embodiments of the present invention, the bacteria is of a species selected from the group consisting of Bacillus subtilis, Bacillus sonorensis, Bacillus licheniformis, Bacilllus firmus, Bacillus megaterium, B. endophyticus, Bacillus endophyticus and Bacillus amyloliquefaciens.

According to embodiments of the present invention, the bacteria are of the species Bacillus subtilis.

According to embodiments of the present invention, the method further comprises purifying the pigment following step (b).

According to embodiments of the present invention, the pigment is comprised in a culture of starch fibers of a legume of a leguminous plant.

According to embodiments of the present invention, the food product is a meat.

According to embodiments of the present invention, the pigment is devoid of material of the leguminous plant.

According to embodiments of the present invention, the food product further comprises a legume of a leguminous plant.

According to embodiments of the present invention, the food product is essentially devoid of material of a legume of a leguminous plant.

According to embodiments of the present invention, the food product is a dry snack.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B. Interaction of B. subtilis with the starch fibers of chickpea milk. Interaction of YC161 (P ₛₚₐₙₖ-gfp) with chickpea milk (CPM) starch fibers that exhibit intense red autofluorescence (A), bacterial fluorescence emission is represented as bright green fluorescence against the faint green auto-fluorescence of the starch fibers, scale bar: 20 μm, and (B) interactions of WT and matrix mutants (ΔtasA and ΔepsH) (intense green fluorescence exhibited by the bacteria stained with SYTO™ 9 dye) with starch fibers (faint green autofluorescence) of CPM, scale bar: 100 μm.

FIGS. 2A-F. Phenotypes of B. subtilis strains in chickpea milk. (A) Pellicle, pigment and colony-type biofilm formations by WT and mutants in chickpea milk (CPM) or CPM agar after 72 h of incubation at 30° C. (B) pellicle formations by pulcherrimin deficient mutants (ΔyvC and ΔcypX) in CPM in the presence or absence of glycerol. (C) CFUs of WT and mutants (ΔtasA, ΔepsH, ΔyvC and ΔcypX) in CPM after 24 h of incubation. (D) growth curve of WT in LB and CPM grown with 150 rpm shaking. The graph shows the means±SEMs of three measurements. *P<0.05 vs. the non-treated controls. (E) interactions of WT and spo0A and sinI mutants (intense green fluorescence exhibited by the bacteria stained with SYTO™ 9 dye) with CPM starch fibers (faint green auto fluorescence). (F) purified pulcherrimin (in methanol) from WT, spo0A and sinI mutants.

FIGS. 3A-D. Survivability of B. subtilis following pasteurization and in vitro gastro-intestinal digestion. (A) Pellicle formation of WT strain in chickpea milk (CPM) at different pH. (B) CFU of B. subtilis 3610 and sinI mutants after subjecting it to in vitro gastro-intestinal digestion. (C) Before GIT treatment, model describing the bacterial-fiber interactions and its survival in gastric and intestinal phases of in vitro digestion system. (D) Effect of WT and sinI mutants to heat treatments. The graph shows the means±SEMs of three measurements. **P<0.01, and ***P<0.001 vs. the non-treated controls.

FIGS. 4A-C. Microscopic visualization and staining of the chickpea starch fibers. (A) Non-fluorescent starch granules. (B) auto-fluorescent starch fibers which are ruptured due to heat due to autoclaving, scale bar: 20 μm, (C) comparative analysis of natural autofluorescence and propidium iodide staining. Propidium iodide stains the starch fibers that are ruptured, while not the intact starch granules as the dye is membrane impermeable. For analysis, unvarying minimal auto-exposure (20 ms) was used which sinks out the natural autoflouresence of CPM, scale bar: 100 μm.

FIGS. 5A-B. Lugol's stain (Pottasium iodide and iodine) confirms that most of the CPM is starch. (A) KI stained CPM, and (B) microscopic visualization shows insoluable fibers as blue color (indication of starch, as KI is orange colour and turns dark blue if it binds to starch) under light microscope. Yellow arrow denotes the auto-fluorescent starch fibers, while the black arrow and all that shows faint blue is the non-fluorescent starch granules.

FIG. 6. Alkaline pH (by addition of KOH to CPM) solubilizes the starch fibers as well as quenches the autofluorescence, thus confirming them as the resistant starch fibers.

FIG. 7. Extensive chaining in CPM by WT strains. GFP expression was monitored for YC161 while WT 3610 was stained with SYTO™ 9 dye.

FIG. 8. Sequential biochemical procedure for extraction of pulcherrimin from CPM.

FIGS. 9A-D. CPM polysaccharides act as an environmental signal for biofilm and pellicle formations. (A) Most of the carbon sources tested formed fragile pellicle in LB supplemented with 0.1 mM manganese. (B and C) pectin (the only soluble dietary fiber in chickpea) formed realistic biofilms and pellicles at 0.1% and 0.5% concentration. (D) pectin-mediated biofilm formation was dependent of Spo0A/SinI pathway.

FIGS. 10A-C. Survivability of B. subtilis following in vitro gastro-intestinal digestion. Colony forming units (CFU) of B. subtilis WT grown in LB or CPM at 30° C. with shaking at 25 rpm for 24 h subjected to gastric and intestinal phases of the in vitro gastro-intestinal digestion.

FIG. 11. Supplementation of chickpea milk (CPM) with chickpea fiber (CPF) induces pellicle and pulcherrimin production. The cells of B. subtilis were incubated for 24 h into CPM supplemented by increasing doses of CPF.

FIG. 12. Differential induction of biofilm formation by dietary fibers. The cells of B. subtilis were grown for 48 hours in LB medium supplemented by different dietary, soluble and insoluble fibers.

FIG. 13. Graph illustrating the growth rate of B. subtilis in different fiber-enriched media.

FIG. 14. Graph illustrating the survivability of B. subtilis grown in different media under in vitro digestion conditions.

FIG. 15. Photographs showing bundling of B. subtilis, as visualized by confocal microscopy.

FIG. 16. Electron micrographs of B. subtilis grown in the presence of different fibers.

FIG. 17. Measurement of tapA expression in the presence of dietary fibers.

### DESCRIPTION OF SPECIFIC EMBODIMENTS OF THE INVENTION

The present invention, in some embodiments thereof, relates to a method of culturing Bacilli bacteria and use of the culture for generating food products and pigments.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

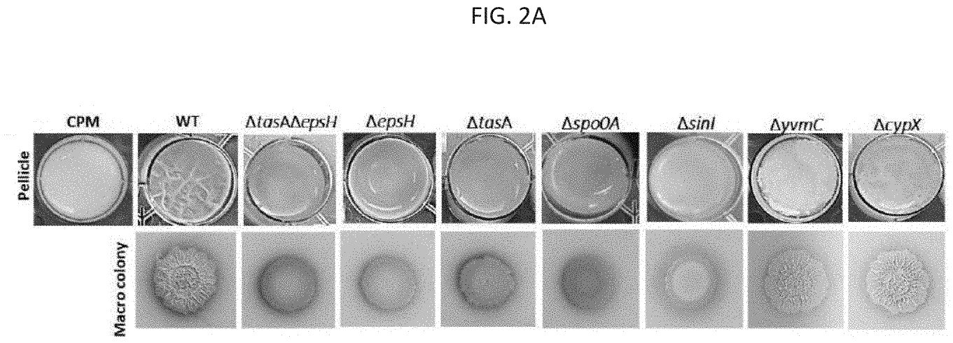

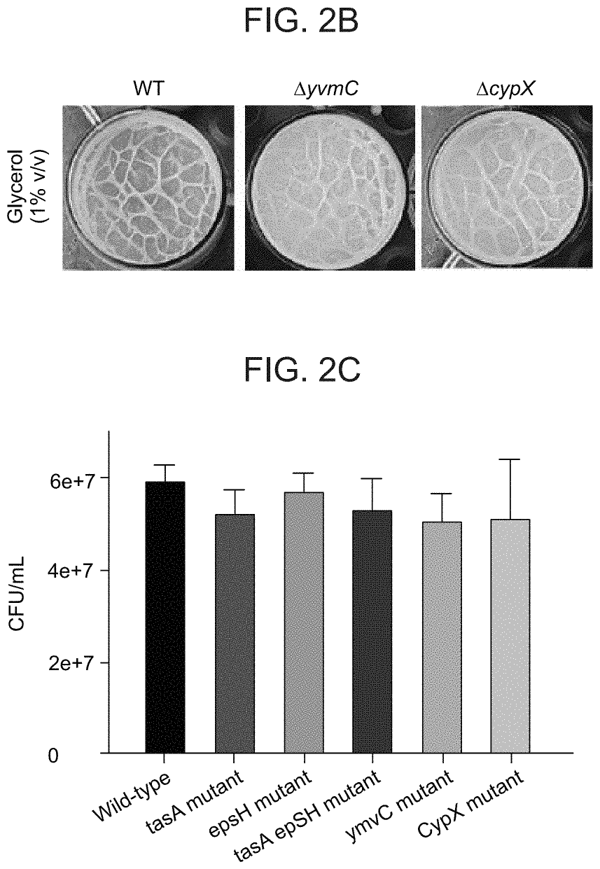

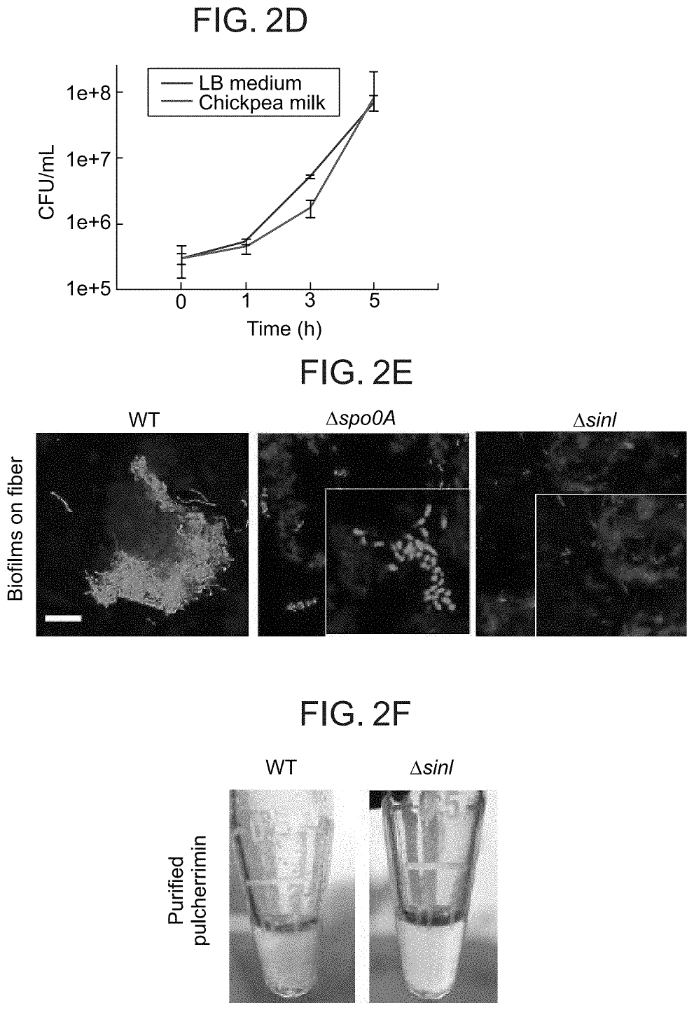

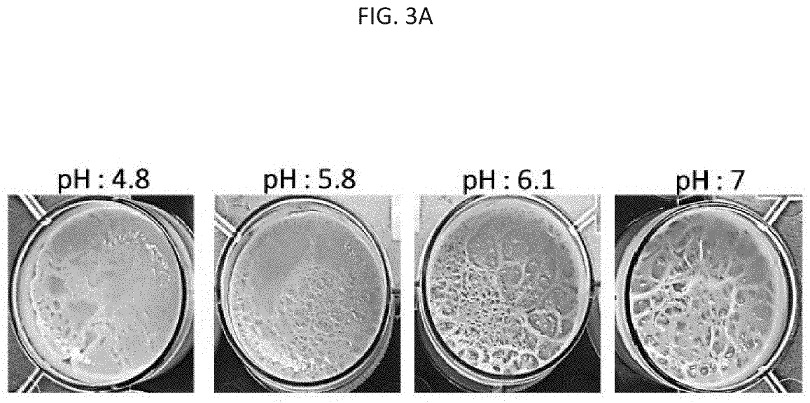

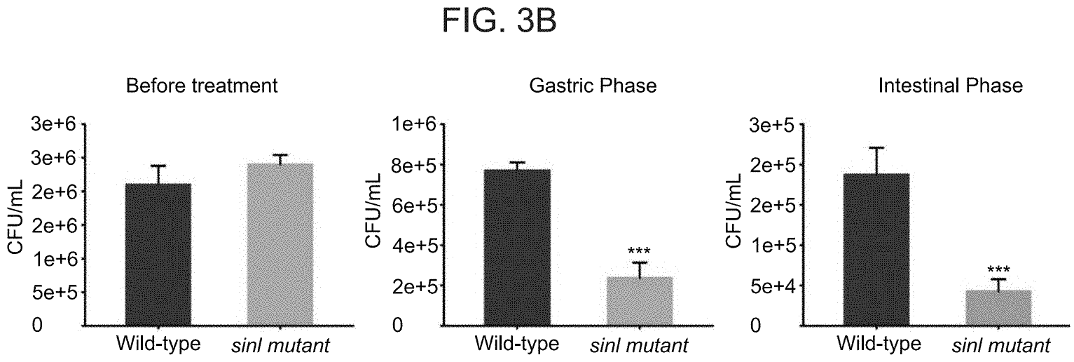

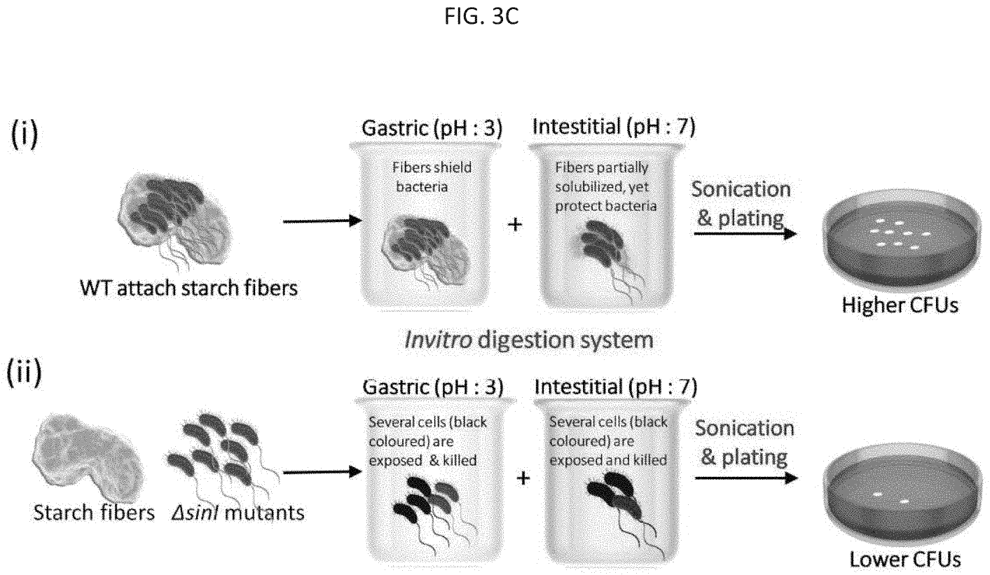

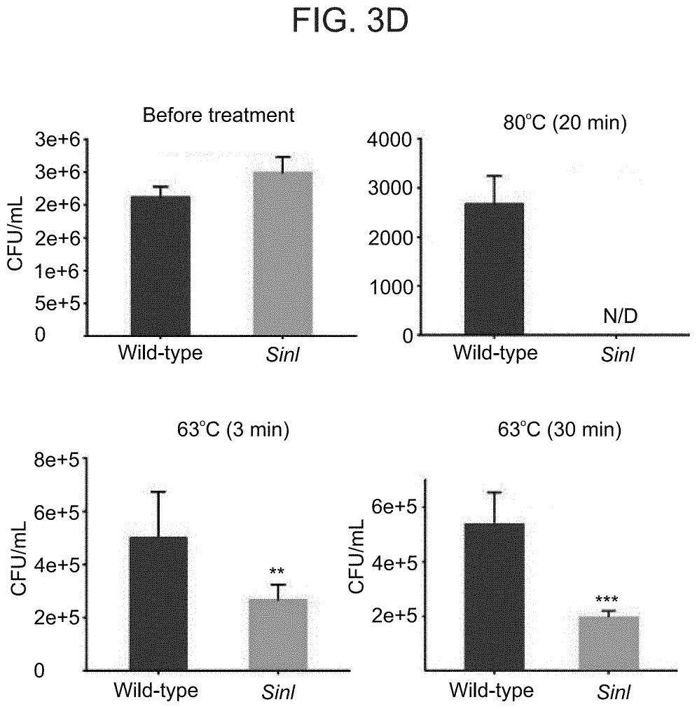

Bacilli exist in natural settings as matrix-enclosed multicellular cells called biofilm. Whilst analyzing the properties of a biofilm of a particular Bacilli species cultured on chickpea starch fibers, the present inventors noticed intriguing morphological changes enabling extraordinary adaptability of the bacterial cells to growth upon the fibers (FIGS. 3A-D). The present inventors propose that dietary starch fibers of additional food products may serve as a natural scaffold for probiotic Bacilli bacteria to colonize and instigate biofilm formation. Whilst reducing the present invention to practice, the present inventors also uncovered that the B. subtilis produce a reddish-pink pigmentation when cultured on the starch fibers (FIG. 2A) and propose that the pigment may be used as a way to color food products, either in the presence or absence of the starch fibers.

Thus, according to a first aspect of the present invention, there is provided a method of culturing bacteria of the Bacilli class, the method comprising:

(a) adding the bacteria to a medium comprising pasteurized starch fibers of a legume, and

(b) culturing the bacteria under conditions that promote generation of a biofilm of the bacteria on the starch fibers, thereby culturing the bacteria.

Bacteria belonging to the class Bacilli, includes the orders Bacillales and Lactobacillales. In one embodiment, the bacteria are of the genus Bacillus, e.g. of the species Bacillus subtilis, Bacillus sonorensis, Bacillus licheniformis, Bacillius firmus, Bacillus megaterium, B. endophyticus, Bacillus endophyticus and Bacillus amyloliquefaciens.

According to a particular embodiment, the species is Bacillus subtilis.

Exemplary strains of Bacillus species contemplated by the present invention include, but are not limited to B. paralicheniformis MS303, B.licheniformis MS310, B. paralicheniformis S127, B. subtilis MS1577, NCIB3610, B. subtilis natto, B. subtilis 168 and B. subtilis PY79.

The term “legume” refers to the seeds or fruit of a leguminous plant.

Examples of leguminous plants which comprise starch fibers on which the bacteria may be cultured include plants of the genus Glycine, plants of the genus Phaseolus, plants of the genus Cicer, plants of the genus Pisum, plants of the genus Lens, plants of the genus Cajanus, plants of the genus Vicia, plants of the genus Arachis, plants of the genus Medicago, plants of the genus Neptunia, plants of the genus Trigonella, and plants of the genus Psophocarpus. Preferred examples thereof include plants of the genus Glycine, plants of the genus Phaseolus, plants of the genus Cicer, plants of the genus Pisum, plants of the genus Lens, plants of the genus Cajanus, plants of the genus Vicia, and plants of the genus Arachis. More preferred examples thereof include plants of the genus Glycine, plants of the genus Phaseolus, plants of the genus Cicer, and plants of the genus Pisum. Further preferred examples thereof include plants of the genus Glycine.

Examples of the plant of the genus Glycine include soybean (Glycine max). Examples of the plant of the genus Phaseolus include common bean (Phaseolus vulgaris). Examples of the plant of the genus Cicer include chickpea (Cicer arietinum). Examples of the plant of the genus Pisum include pea (pea sprout) (Pisum sativum). Examples of the plant of the genus Lens include lentil (Lens culinaris). Examples of the plant of the genus Cajanus include pigeon pea (Cajanus cajan). Examples of the plant of the genus Vicia include broad bean (Vicia faba). Examples of the plant of the genus Arachis include peanut (Arachis hypogaea). Examples of the plant of the genus Medicago include alfalfa (Medicago sativa). Examples of the plant of the genus Neptunia include water mimosa (Neptunia oleracea). Examples of the plant of the genus Trigonella include fenugreek (Trigonella foenum-graecum). Examples of the plant of the genus Psophocarpus include

Goa bean (Psophocarpus tetragonolobus).

In a particular embodiment, the legume is chickpea.

As used herein, the phrase “starch fiber” refers to a polysaccharide comprising at least 3 sugar monomers. The size of fibers may vary between 10-1000 μm.

Starch fibers typically auto-fluoresce when visualized under a confocal laser scanning microscope. Propidium iodide (PI) staining may be used to confirm the presence of starch fibers since it selectively stains the auto-fluorescent starch particles and does not penetrate the membranes of starch granules.

Methods of releasing (or enhancing the amount of) starch fibers from legumes include heating (e.g. cooking) for an amount of time such that autofluorescence may be observed under a fluorescent microscope. Thus, for example chickpeas may be cooked for about 20 minutes to about 60 minutes.

The starch fibers may be retrieved from fresh legumes, frozen legumes, dried legumes or canned legumes.

The legumes may be treated prior to heating to enhance the process of starch fiber release. Thus, the legume may be soaked, crushed, milled and/or homogenized prior to heating.

Optionally, the heated legumes may be further treated prior to use. Exemplary treatment methods include filtration, homogenization and extraction.

Once the starch fibers are released from the legume, they may be used as part of a culture medium. The culture medium is pasteurized or sterilized.

In one embodiment, the culture medium is a chickpea milk i.e. a liquid chickpea suspension, as further described in the methods section herein below, which comprises chickpea starch fibers.

In another embodiment, the culture medium is a medium known for culturing bacillus, to which isolated (exogenous) chickpea fibers have been added. Additional methods of isolating chick pea fibers are known in the art—see for example US Patent Application No. 20200390131, the contents of which are incorporated herein by reference.

In one embodiment, chickpea fibers are isolated using the following steps:
i. Chickpea material is subjected to an oil separation. The oil separation can be performed with a solvent such as, but not limited to, hexane, petroleum ether, or ethanol;
ii. Separation of starch and fibers is then carried out on the basis of their density. Examples of growth media which can be used for culturing bacillus bacteria (to which the chickpea fibers may be added) include, but are not limited to LB, LBGM, milk and MRS. Additional examples of growth media are provided in US Patent Application No. 2020-0190463-A1.

Thus, the present invention contemplates growth media which are fortified with chickpea fibers (e.g. dried chickpea fibers). In one embodiment, the growth medium is chickpea milk fortified with exogenous (i.e. isolated) chickpea starch fibers.

The term “pasteurization” as used herein refers to a heating process that results in the reduction of the number of viable pathogens in the culture medium so they are unlikely to cause disease when consumed by a human (assuming the pasteurized product is stored as indicated). In one embodiment, the pasteurization does not affect the taste or texture of the product.

As used herein, the term “sterilization” refers to a process that eliminates or removes all forms of fungi, bacteria, viruses, spore forms, or other microbiological organisms present in the culture media, or other suitable items.

Prior to addition of the bacteria to the culture medium, the pH may be adjusted.

In one embodiment, the pH of the culture medium is higher than 6.

The bacteria, which is added to the culture medium, may be in a starter culture, as known in the art.

Culturing of the bacteria in the culture medium is carried out under conditions that promote the generation of biofilm on the starch fibers present in the medium.

In one embodiment, the culture medium is a liquid medium.

In another embodiment, the culture medium is a solid medium (e.g. further comprises a gelling agent).

Examples of gelling agents contemplated by the present invention include, but are not limited to agar, guar gum, xanthan gum, locust bean gum, gellan gum, polyvinyl alcohol, alkylcellulose, carboxyalkylcellulose and hydroxyalkylcellulose.

As mentioned, the culture conditions are such that they allow for generation of a biofilm on the fibers.

The present inventors have uncovered particular components of a culture medium that are important for biofilm generation of bacteria being of the genus Bacillus (e.g. of the species B. subtilis). Thus, the present inventors propose that the medium used for culturing the B. subtilis further comprises manganese. In another embodiment, the medium further comprises glycerol. In still another embodiment, the medium further comprises dextrose. In still another embodiment, the medium used for culturing comprises both manganese and dextrose.

Other conditions of the culture that may be altered to enhance generation of a biofilm include, but are not limited to environmental parameters such as pH, nutrient concentration, co-culture of additional bacteria and temperature.

In one embodiment, the culturing is carried out in a bioreactor.

As used herein, the term “bioreactor” refers to an apparatus adapted to support the growth of bacteria on the starch fibers.

The bioreactor will generally comprise one or more supports for the starch fibers, and wherein the support is adapted to provide a significant surface area to enhance the formation of biofilm over the starch fibers. The bioreactors of the invention may be adapted for continuous throughput.

It will be appreciated that when the biofilm is generated in a bioreactor system, the conditions of the culture can be altered by altering the microfluidics (e.g. sheer stress) of the system.

As mentioned, agents or conditions are selected that bring about an advantageous change in a property of the biofilm. In one embodiment, the property is the amount of biofilm. In one embodiment, the property is the thickness of biofilm. In another embodiment, the property is the density of the biofilm. In yet another embodiment, the property is the rate in which the biofilm is formed. In still another embodiment, the property is the amount of additional bacteria which is incorporated into the biofilm. In still another embodiment, the property is the resistance to temperature and/or pH. In still another embodiment, the property is the amount of pigment generated (or secreted) by the bacteria, as further described herein below.

The culturing of this aspect of the present invention may be carried out in the presence of additional agents that serve to increase propagation of the B. subtilis bacteria and/or enhance biofilm formation. Such agents include for example acetoin.

The amount of acetoin and the timing of addition may be altered so as to promote optimal biofilm production. In one embodiment, about 0.01-5% acetoin is used. In another embodiment, about 0.01-4% acetoin is used. In another embodiment, about 0.01-3% acetoin is used. In another embodiment, about 0.01-2% acetoin is used. In another embodiment, about 0.01-1% acetoin is used. In another embodiment, about 0.01-0.5% acetoin is used.

In one embodiment, about 0.05-5% acetoin is used. In another embodiment, about 0.05-4% acetoin is used. In another embodiment, about 0.05-3% acetoin is used. In another embodiment, about 0.05-2% acetoin is used. In another embodiment, about 0.05-1% acetoin is used. In another embodiment, about 0.05-0.5% acetoin is used.

In one embodiment, about 0.1-5% acetoin is used. In another embodiment, about 0.1-4% acetoin is used. In another embodiment, about 0.1-3% acetoin is used. In another embodiment, about 0.1-2% acetoin is used. In another embodiment, about 0.1-1% acetoin is used. In another embodiment, about 0.1-0.5% acetoin is used.

The cultures of this aspect of the present invention are propagated for a length of time sufficient to generate a biofilm on the starch fibers which incorporates the Bacillus bacteria.

In one embodiment, the conditions are selected such that the amount of biofilm produced by the Bacillus is enhanced and the amount of a pigment secreted from the biofilm is enhanced. Alternatively, the conditions are selected such that the amount of pigment secreted by the Bacillus is enhanced.

According to a particular embodiment, the color of the pigment is dark brown/red.

Preferably, the Bacillus bacteria are cultured for at least 6 hours, 12 hours, 24 hours, 48 hours, 3 days, 4 days, 5 days, 6 days, 7 days, 8 days, 2 weeks, 3 weeks or longer to allow for sufficient quantities of a pigment to be generated and/or for sufficient quantities of biofilm to be generated.

In one embodiment, the Bacillus bacteria are cultured at a temperature between 20-40° C., more preferably between 23-32° C.—for example at about 30° C.

As mentioned, the Bacillus bacteria generate a reddish brown pigment when cultured on starch fibers of legumes.

Thus, according to another aspect of the present invention there is provided a method of generating a pigment comprising:

(a) culturing bacteria belonging to the genus Bacillus on a medium comprising pasteurized starch fibers of a legume of a leguminous plant under conditions that allow the secretion of the pigment into the medium; and

(b) collecting the medium.

Conditions that allow for the secretion of pigment into the medium are typically those that promote formation of a biofilm, as described herein above.

Following sufficient time in culture, the pigment may be isolated from the culture.

Preferably, the method for isolating the pigment involves a step of removing the culture medium from the Bacillus cells. This extracellular fraction of the liquid fermentation medium is also termed the supernatant and this fraction can be separated from the Bacillus cellular fraction by e.g. centrifugation or filtration, or indeed by any other means available for obtaining a liquid fraction essentially without any bacterial cells present therein.

In particular embodiments of the invention, the purification comprises at least one size fractionation step. Preferably, this size fractionation step is performed on the extracellular fraction. This size fractionation step may ensure that every component of the composition has a molecular weight of at least a given value. The size fractionation step may be any size fraction known to the skilled person, for example ultracentrifugation, ultrafiltration, microfiltration or gel-filtration. Thus in a particular embodiment of the invention, the pigment is purified from a liquid growth medium by a method involving one or more purification steps selected from the group consisting of ultracentrifugation, ultrafiltration, microfiltration and gel-filtration. Preferably, the purification step(s) are selected from the group consisting of ultrafiltration, microfiltration and ultracentrifugation, even more preferably from the group consisting of ultrafiltration and microfiltration.

Ultrafiltration is a membrane process where the membrane fractionates components of a liquid according to size. The membrane configuration is normally cross-flow wherein the liquid containing the relevant components are flowing across the membrane. Some of the liquid, containing components smaller than the nominal pore size of the membrane will permeate through the membrane. Molecules larger than the nominal pore size will be retained. The desired product may be in the retentate or the filtrate. If the ultrafiltration is performed in order to prepare a composition, wherein every agent within the composition has a molecular weight above a given value, the desired product is in the retentate. If a serial fractionation is made, the product may be in the retentate or filtrate.

Microfiltration is a membrane separation process similar to UF but with even larger membrane pore size allowing larger particles to pass through.

Gel filtration is a chromatographic technique in which particles are separated according to size. The filtration medium will typically be small gel beads which will take up the molecules that can pass through the bead pores. Larger molecules will pass through the column without being taken up by the beads.

Gel-filtration, ultrafiltration or microfiltration may for example be performed as described in R Hatti-Kaul and B Mattiasson (2001), Downstream Processing in Biotechnology, in Basic Biotechnology, eds C Ratledge and B Kristiansen, Cambridge University Press) pp 189.

In another embodiment the pigment in the medium may be isolated by precipitation, such as precipitation with alcohol, such as ethanol and/or chromatographic methods. This may for example be performed essentially as described in WO2003/020944. It is also contemplated within the invention that the pigment is isolated by sequentially performing two or more of above-mentioned methods. By way of example the pigment may be isolated by first performing a size fractionation step followed by precipitation.

Other methods for isolating the pigment of this aspect of the present invention are also contemplated by the present inventors including but not limited to HPLC.

The pigment which is generated by the Bacillus bacteria may be used to color a food product.

Thus, according to another aspect of the present invention, there is provided a method of coloring a food product comprising combining a pigment generated by Bacillus subtilis with the food product under conditions that alter the color of the food product, thereby coloring the food product.

In one embodiment, the pigment is added in sufficient quantities such that it provided a red/brown color to a food product. The food product may comprise an animal derived meat protein or a plant-based protein that is used as a meat substitute.

In one embodiment, the pigment is added to a plant-based meat substitute in sufficient quantities such that it obtains a color of a raw, uncooked meat. In another embodiment, the pigment is added to a plant-based meat substitute such that it obtains the color of a cooked meat.

The food product typically comprises vegetable proteins or animal derived proteins. Animal derived protein materials that may be utilized include, but are not limited to, collagen protein, casein or caseinate proteins, and whey protein albumin. Vegetable protein materials which may be utilized include, but are not limited to, gluten materials and soy protein materials. Most preferably the protein in the protein containing material is a soy protein material such as soy protein isolate, soy protein concentrate, soy flour, soy flakes, or mixtures thereof, where the soy protein material preferably contains at least about 50% soy protein. These protein materials are commercially available from various manufacturers, for example, soy protein isolates that may be used in the invention include SUPRO 500E, SUPRO EX 31-33, and SUPRO 515-516, which can be purchased from Protein Technologies International, Inc., Checkerboard Square, St. Louis, Mo. 63164.

The protein containing material may also include adjuncts, including, but not limited to, starches, gums, and fibres, and mixtures thereof. The adjuncts may be included to impart various functionalities to the protein containing material to improve the meat-like characteristics of the protein containing material. For example, starch may be included in the protein containing material to increase the viscosity and gel forming capability of the protein containing material when the protein containing material is hydrated. Gums may be included in the protein containing material to enhance the flowability of the protein containing material. Fibres may be included in the protein containing material to enhance the structure of the protein containing material when hydrated.

The pigment may be added to the protein containing material in an aqueous solution, where the pigment is diluted in water before being added to the protein containing material. In one embodiment, the pigment is diluted in a small quantity of water to form an aqueous solution of the pigment, which is then dispersed with the protein containing material in a quantity of water for hydrating the protein containing material. Alternatively, a dry pigment and a protein containing material may be dispersed together in water for hydrating the protein containing material.

Preferably the colored hydrated protein containing material contains from about 0.0005% to about 0.005%, by weight, of the pigment.

Simultaneously in conjunction with hydration and treatment with the pigment, or after being hydrated and treated with the pigment, the protein containing material may be texturised by any of a number of known methods for texturising protein materials to provide a meat-like texture to the protein containing material. For example, known processes for texturising protein materials include creating bundles of spun fibres of protein material after hydration of a protein material; extruding a hydrated protein material at a controlled pH, where the fat content of the protein material is minimised; and forming a textured granulated gel of a hydrated protein material.

The colored protein containing material, optionally texturised and flavoured, may be used as a meat analogue or a meat extender (i.e. increases the volume/amount of an animal-based product). In one aspect of the invention, the colored protein containing material may be formed into patties or stuffed into casings by itself to form a meat analogue patty or sausage. The meat analogue patties and sausages may be cooked, for example by frying or broiling, at temperatures, and for a time period, effective to cook the meat analogue, for example from about 50° C. to about 260° C.

According to a particular embodiment, the pigment is added after it has been isolated from the bacterial culture.

In another embodiment, the pigment is added together with the bacterial culture described herein (i.e. together with the starch fibers of a legume, e.g. chickpea). This is advantageous in two respects—the bacteria of the bacterial culture may be probiotic (e.g. B. subtilis) and the starch fibers of the legume add nutritional value to the food product.

According to another aspect of the present invention, there is provided a food product comprising a culture which comprises bacteria belonging to the class Bacilli and a medium comprising starch fibers of a pasteurized legume of a leguminous plant.

The proportion of Bacilli bacteria to starch fibers of a legume may be such that the starch fibers serve to protect the probiotic Bacilli bacteria in the acid environment of the gastrointestinal tract of a person. Thus the proportion of Bacilli bacteria to starch fibers of the legume may be such that the starch fibers enhance survival of the probiotic Bacilli bacteria in an acid environment.

Examples of food products to which the culture may be added include breakfast cereals, dry snacks, pasta, cakes, chips, meat analogues.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Bacterial Strains and Culture Conditions

All B. subtilis strains were cultured in Lysogeny broth (LB) comprising 10 g of tryptone, 5 g of yeast extract, and 5 g of NaCl or on solid LB medium supplemented with 1.5% agar. For generating the starter cultures, initially, the strains were streaked on LB agar plates from the glycerol stocks maintained at −80° C. and incubated overnight at 37° C. A colony from the overnight LB agar plate was inoculated in fresh LB and grown at 37° C. for 5 hours with 150 rpm. The resultant culture was used as the starter culture for all the experiments.

Chickpea Milk Preparation

Approximately, 187.5 g of Kabuli-type chickpea (Cicer arietinum) seed were soaked in 1.2 L of distilled water (DW) and incubated for 12 h at room temperature. Following incubation, fresh DW was replaced and the seeds were crushed using a blender. The liquid chickpea seed suspension, chickpea milk (CPM) was boiled for 10 minutes, filtered using a sieve, following which the pH was adjusted with lemon juice (for pH: 4.8, 5.8 and 6.1) or 1 mM NaOH (for pH: 7). Finally, CPM was autoclaved and stored for further assays.

Macroscopic Assessment of Biofilms

Starter cultures of the required strains were prepared as previously described. For pellicle formation assays, 5 μl of the bacterial suspensions (5×10⁵ CFU/mL) were pipetted into 4 mL of CPM in a 12 well polystyrene plates, while for colony-type biofilm assays, 3 μl of the bacterial suspensions were spotted on a CPM solid medium supplemented with 1.5% agar. All the plates were incubated at 30° C. for 72 hours and images were captured using either a regular camera or a Zeiss Stemi 2000-C microscope with an axiocam ERc 5s camera (Zeiss, Germany).

Microscopic Analysis

For visualization of CPM components, propidium iodide (Promega, USA) and Lugol's stain was used, while for visualization of B. subtilis interactions with the CPM fibers, fluorescently tagged B. subtilis YC161 strain, that constitutively expressed the green fluorescent protein (GFP) was used. The samples were processed and stained as previously described [4] and visualized under a confocal laser scanning microscope (CLSM) (Leica, Wetzler, Germany). Strains that did not express GFP were stained with SYTO™ 9 dye from the Filmtracer live/dead biofilm viability kit (Promega, USA) in conformity with the guidelines.

In Vitro Digestion System

The survivability of B. subtilis WT and ΔsinI mutants in an acidic environment was monitored by an in vitro digestion system [2]. Briefly, the starters were diluted 1:100 into LB or CPM and incubated at 30° C. with shaking at 25 rpm for 24 h, following which the samples were subjected to in vitro gastro-intestinal digestion procedures as previously described [2]. Following in vitro digestion, samples were sonicated for 2 min (10 s pulse on/off) at 4° C. with 40% amplitude, plated on LB agar plates and incubated overnight at 37° C. Following incubation, the colonies were enumerated by colony forming units (CFU) method.

Assessment of Bacterial Sensitivity to Heat Treatment

The sensitivity of B. subtilis WT and sinI mutants to heat was monitored by pasteurization. Starter cultures of the bacterial strains were prepared as previously described. The cultures were then diluted 1:100 in CPM and grown for 24 h at 30° C. with 25 rpm shaking. Following incubation, the samples were then heat treated in three separate batches or tubes (63° C. for 3 min, 63° C. for 30 min, and 80° C. for 20 min) in a water bath. Immediately after pasteurization, the samples were sonicated for 2 min (10 s pulse on/off) at 4° C. with 40% amplitude with an Ultrasonic processor (Sonics, VCX 130, Newtown, USA), diluted, plated and incubated overnight at 37° C. The number of surviving cells was enumerated by CFU method.

Statistical Analysis

All experiments were conducted in triplicate, and results are expressed as means±standard deviations. Statistical significance was determined by pair-wise testing using the Students' t-test, and was accepted for p values of *p<0.05, **p<0.01, and ***p<0.001.

### Example 1

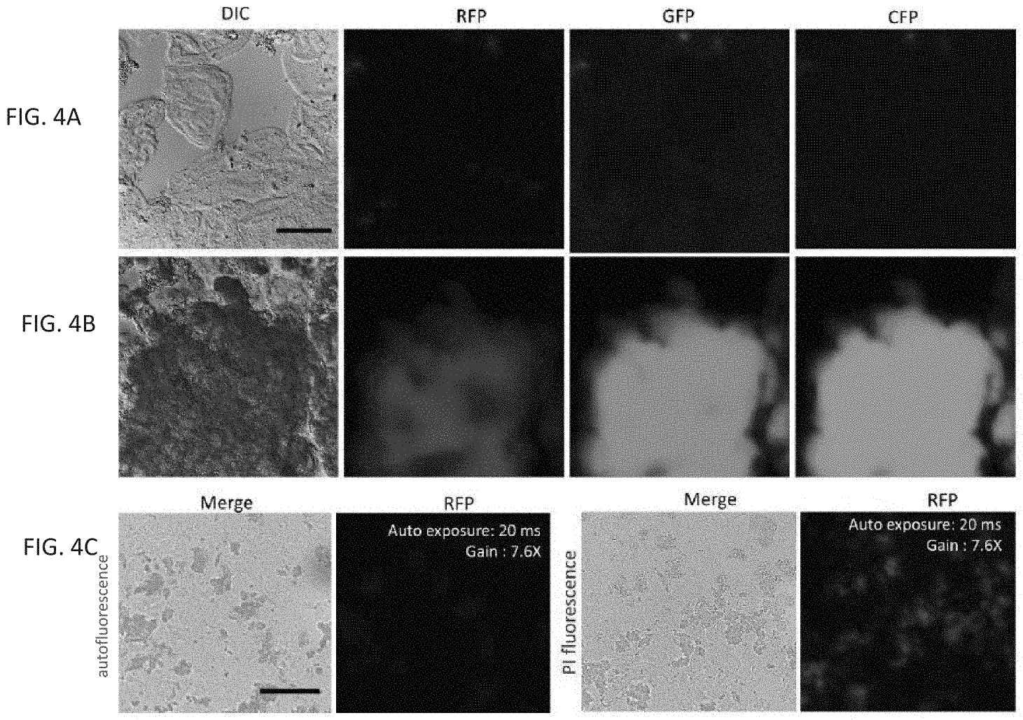

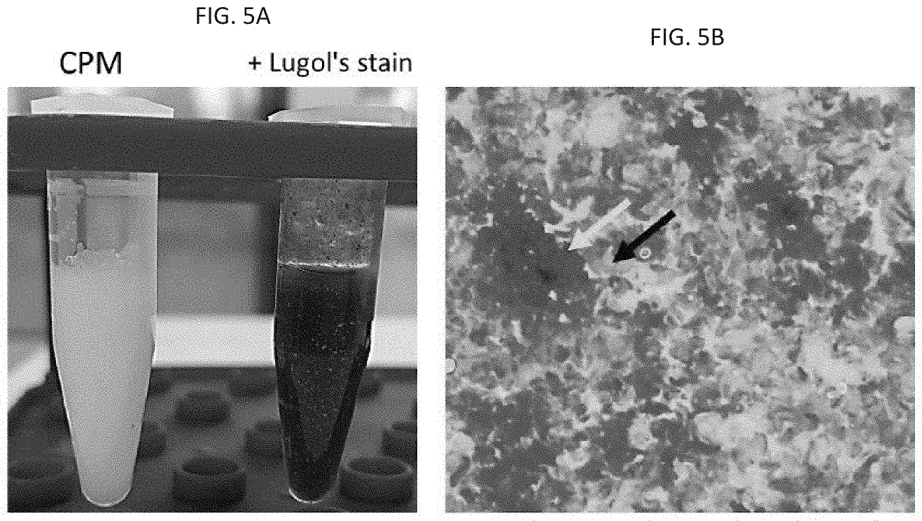

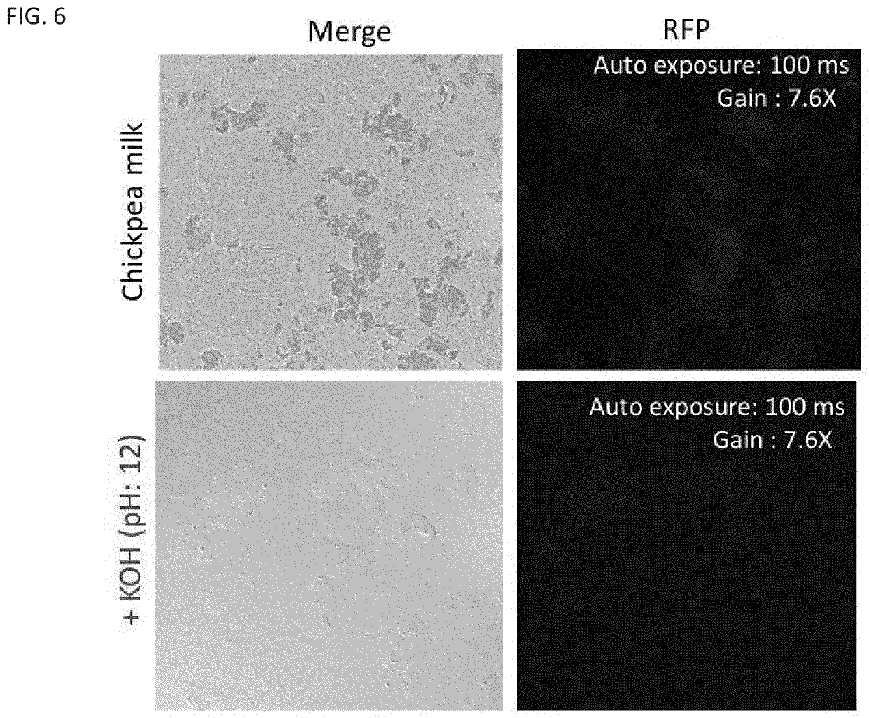

Initial microscopic analysis of the CPM medium revealed the presence of insoluble components that was categorized as non-fluorescent (FIG. 4A) or auto-fluorescent fibers (FIG. 4B). Interestingly, the non-fluorescent fibers of CPM resembled the starch granules (FIG. 4A) consistent with previous reports [17, 18]. Likewise, presence of auto-fluorescent starch in dry-sprayed chickpea seeds was microscopically demonstrated by Susan et al, 2013 [15]. Here, the aggregation of auto-fluorescent starch fibers in liquid-phase state was observed. This aggregation was irreversible and happened during starch gelatinization due to high temperature heating (boiling) and subsequent cooling (FIG. 4B). Lugol's and propidium iodide (PI) staining confirmed that the fibers were indeed starches (FIGS. 4C, 5A and 5B). The former stained all of the starch particles in CPM, while the latter selectively stained the auto-fluorescent starch particles and not the granules, as PI does not penetrate the membranes of the starch granules (FIG. 4C). In addition, an enhanced fiber solubility and loss of auto-fluorescence was observed at higher pH following the KOH addition (FIG. 6), thus confirming the auto-fluorescent fraction as the resistant starch particles.

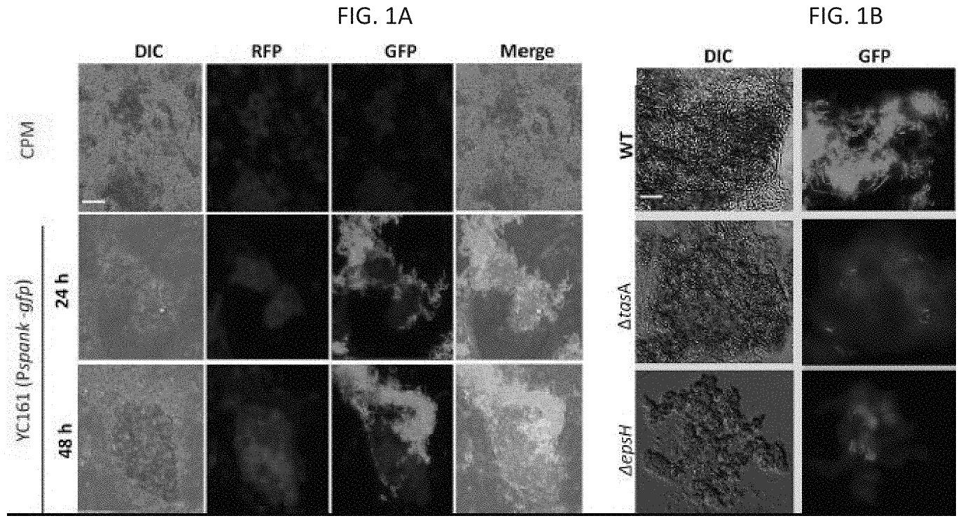

In order to determine whether these starch fibers might serve as a natural scaffold for bacteria to grow as biofilms, the fluorescently tagged wild type B. subtilis strain (YC161 (P ₛₚₐₙₖ-gfp)), that constitutively expresses the green-fluorescent protein (GFP), were cultured in CPM. Interestingly, it was found that B. subtilis YC161 selectively colonize the autofluorescent starch fibers apparently through tight interactions (FIG. 1A). These intriguing interactions indicate that Bacillus cells could preferentially colonize the starch fibers. However, it was noticed that the bacterial cells formed also suspended bundles that did not attach to these fibers, and demonstrated extended chaining in CPM (FIG. 7). Z-stack analysis by CLSM confirmed that this intriguing bacterial-fiber interaction were rigid and dynamic.

Since B. subtilis biofilm is basically reliant on extracellular polymeric substance matrix, the growth of strains (ΔtasA, and ΔepsH) that harbored mutants in matrix operons was assessed. None of them adhered to the auto-fluorescent fibers nor formed biofilm bundles in CPM (FIG. 1B), thus affirming the involvement of matrix production in this bacteria-fiber interaction. Biofilm forming ability of B. subtilis cells was further characterized phenotypically in standing cultures; obviously, the WT cells formed robust pellicle and colony type biofilm in CPM. As expected, matrix mutants (ΔtasA, ΔepsH, and the double mutant) could not form either type of biofilm in CPM (FIG. 2A). Since matrix mutants failed to form biofilm, it was assumed that the observed phenomenon could be related to the Spo0A/SinI regulatory pathway. The strains harboring deletion mutations in either spo0A or sinI did not form pellicle or colony biofilm, nor attachment to the starch fibers (FIGS. 2A and 2E). The best possible explanation of the observed results is that in the absence of SinI, SinR represses the biofilm phenotypes in CPM. However, 4spo0A mutants formed eccentric cell clusters or bunches in CPM, while not adhering to the starch fibers (FIG. 2E). It may be postulated that in the absence of Spo0A, cells might activate a different pathway that triggers cell clustering.

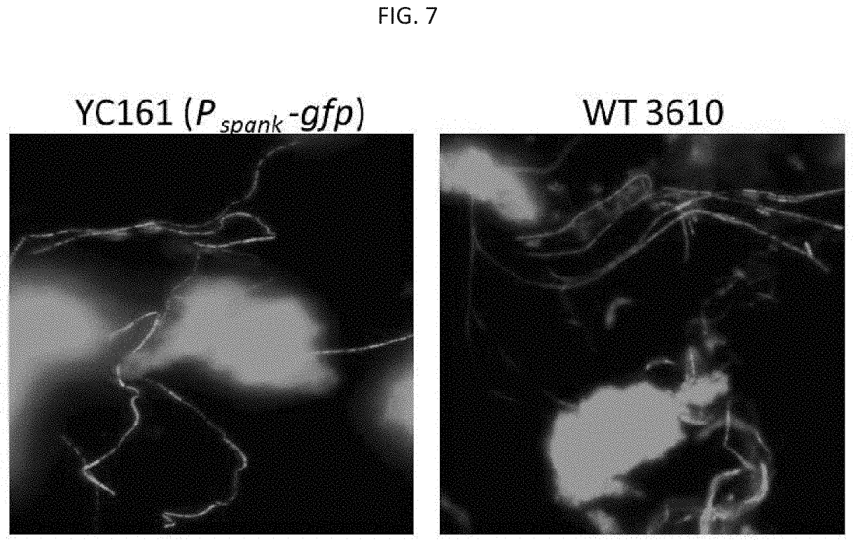

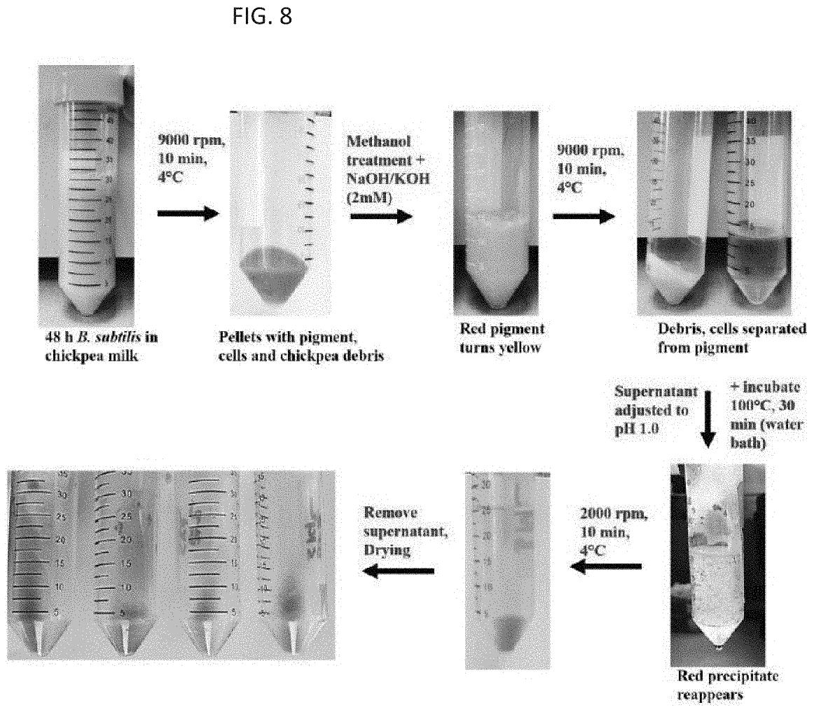

Moreover, it was further observed that the WT cells of B. subtilis produce an inquisitive reddish-pink pigmentation in CPM that was somewhat contemporaneous in the epsH, tasA and spo0A mutants as well (FIG. 2A). Intriguingly, the sinI mutant cells refrained from producing the pigment. B. subtilis produces assorted spectrum of pigments that may or may not be correlated with biofilm formation [19]. Pulcherimin is one such pigment that B. subtilis produces during biofilm mode of growth [19]. Pigment production by Bacilli is also recognized as a survival tactics exhibited by the vegetative cells against external stressors. The biochemical traits of pulcherrimin was close to the pigment observed (FIG. 8). Pulcherrimin-deficient mutants lack the enzymes YvmC (Cyclo(L-leucyl-L-leucyl) synthase) and CypX (Pulcherriminic acid synthase). These convert two tRNA-molecules of leucine to pulcherriminic acid (PA), which in turn binds to iron (Fe³⁺) to form pulcherrimin. The mutants failed to produce the pigment in solid and liquid CPM (FIG. 2A). It was also reported that pulcherrimin could be induced by addition of glycerol or starch [20]. When tested in CPM, glycerol apparently induced robustness in the pellicles although did not show any enhancement in the qualitative pigmentation levels in WT strain (FIG. 2B). However, the glycerol failed to induce the pigmentation in pulcherrimin-deficient strains (FIG. 2B). Therefore, the pigment was confirmed as pulcherrimin. Possibly, pulcherrimin production in CPM is attributed to bacterial-starch interactions, suggesting starch might be a carbon source for triggering this phenotype.

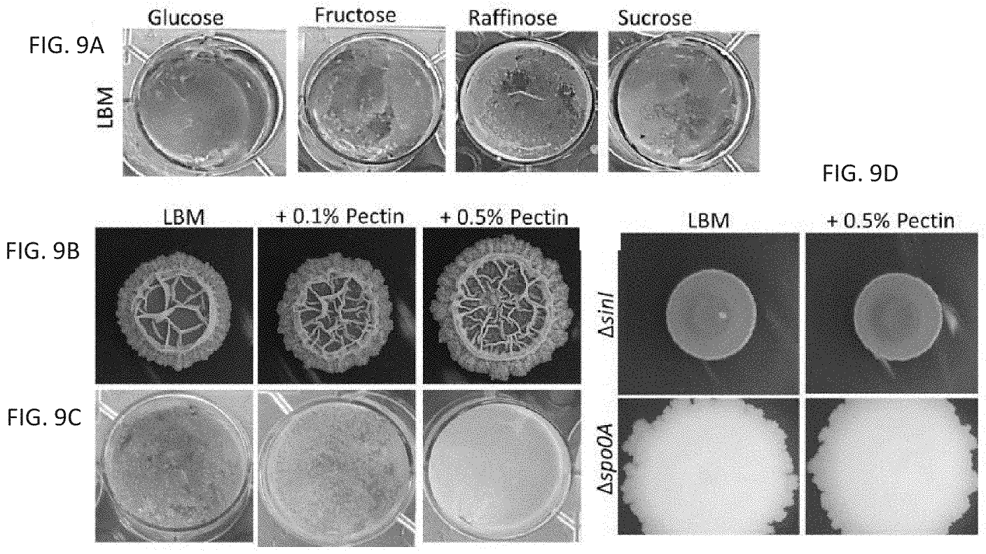

Next, pulcherrimin was further purified and compared between WT and sinI mutants. The results showed no pigment production in ΔsinI mutants (FIG. 2F). The growth of all the other tested strains had no significant change (FIG. 2C), and the growth curve of WT in CPM was analogous to its growth in LB in shaking cultures (FIG. 2D). The results suggest that all the tested mutants displayed defects in matrix synthesis without hampering the growth in CPM, while only ΔsinI mutant showed defects in pulcherrimin synthesis. Additional challenge was to identify the signaling molecule (presumably carbon source) that might be responsible for the biofilm or pigment formation. FIGS. 9A-D indicate that the soluble fiber, pectin, and no other tested sugars (glucose, fructose, sucrose and raffinose) is involved in biofilm and pigment formation.

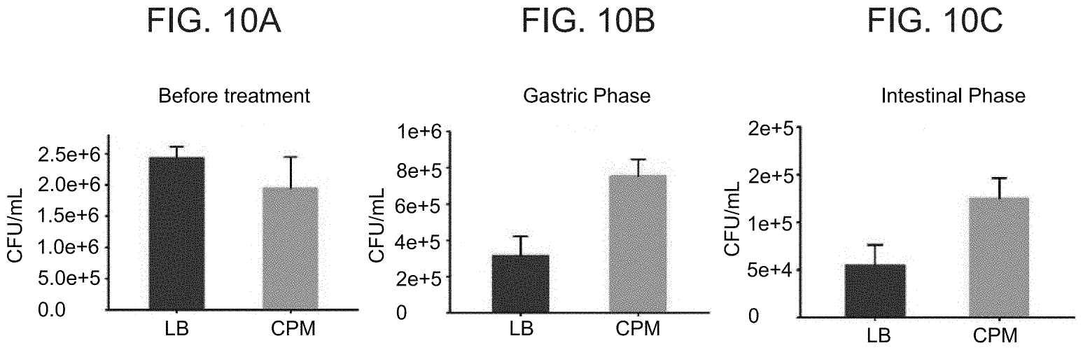

Next, the ability of the WT strain to grow in different pHs was analyzed. Alkaline pH enhanced the robustness in the pellicles while pigment was best at lower pH (FIG. 3A). The viability of WT strain in LB medium following in vitro digestion system as described previously was analyzed [2]. A significant loss of viability in cells grown in LB compared to cells grown in CPM was noted (FIGS. 10A-C). WT cells grown in CPM were resilient to hostile effects during in vitro digestion system and exhibited higher survival rates than ΔsinI mutants in both gastric and intestinal phases (FIG. 3B, and FIGS. 10A-C). Higher bacterial survival rates in CPM may be due to its ability to interact with the fibers. To substantiate further, the survival rates of ΔsinI mutants that existed as individual cells was tested in CPM. Interestingly, a drastic reduction in the viability of ΔsinI mutants was observed confirming potential role of CPM starch fibers in protecting the bacteria as depicted in FIGS. 3B and 3C.

Milk and dairy-free milk products like CPM need to undergo thermal processing like pasteurization before packaging. Probiotics are sensitive to heat and do not endure this procedure [22]. Techniques like microencapsulation have been shown to improve the viability and are often recommended [9]. It was anticipated that the prebiotic fibers in CPM might shield bacteria from physical stresses such as heat treatment. Consequently, the WT cells showed remarkable survivability, while amount of the ΔsinI mutant cells were significantly reduced following exposure to heat treatment (FIG. 3D). Higher survival rates in the WT strain ascribe to its ability to form biofilms on the starch fibers that in turn shield it from heat-stress. The ΔsinI mutant that existed as free cells in CPM were highly susceptible to heat treatments. Overall, it has been found that the interaction of B. subtilis cells with starch fibers in CPM provide ideal conditions to withstand varied stressors.

### REFERENCES

1. Vlamakis, H. et al. Sticking together: building a biofilm the Bacillus subtilis way. Nat Rev Microbiol. 11, 157-168 (2013).
2. Yahav, S. et al. Encapsulation of beneficial probiotic bacteria in extracellular matrix from biofilm-forming Bacillus subtilis. Artif. Cells Nanomed. Biotechnol. 27, 1-9 (2018).
3. Van Gestel, J., Vlamakis, H., Kolter, R. From cell differentiation to cell collectives: Bacillus subtilis uses division of labor to migrate. PLoS Biol. 13, e1002141 (2018).
4. Pasvolsky, R. et al. Butyric acid released during milk lipolysis triggers biofilm formation of Bacillus species. Int. J. Food Microbiol. 181, 19-27 (2014).
5. Cairns, L. S., Hobley, L., & Stanley-Wall, N. R. Biofilm formation by Bacillus subtilis: new insights into regulatory strategies and assembly mechanisms. Mol Microbiol. 93, 587-598 (2014).
6. Beauregard, P. B. et al. Bacillus subtilis biofilm induction by plant polysaccharides. PNAS. 110, 1621-1630 (2013).
7. Piewngam, P. et al. Pathogen elimination by probiotic Bacillus via signalling interference. Nature. 562, 532-537 (2018).
8. Lamya, R. et al. Effect of Bacillus subtilis strains on intestinal barrier function and inflammatory response. Front Microbiol, 10, 564 (2019).
9. Elshaghabee, F. M. F. et al. Bacillus as potential probiotics: Status, concerns, and future perspectives. Front Microbiol. 8: 1490 (2017).
10. Markowiak, P. & Śliżewska, K. Effects of probiotics, prebiotics, and synbiotics on human health. Nutrients, 9, 1021 (2017).
11. Ranadheera, C. S. et al. In vitro analysis of gastrointestinal tolerance and intestinal cell adhesion of probiotics in goat's milk ice cream and yogurt. Food Res. Int. 49, 619-625 (2012).
12. Terpou, A. et al. Enhanced probiotic viability and aromatic profile of yogurts produced using wheat bran (Triticum aestivum) as cell immobilization carrier. Process Biochem. 55, 1-10 (2017).
13. Terpou, A. et al. Novel frozen yogurt production fortified with sea buckthorn berries and probiotics. LWT Food Sci. Technol, 105, 242-249 (2019).
14. Swati, S. S. Tyagi, K. & Anurag, R. K. Plant-based milk alternatives an emerging segment of functional beverages: a review. J Food Sci Technol. 53, 3408-3423 (2016).
15. Susan, M. T. et al. Nutritional profile and carbohydrate characterization of spray-dried lentil, pea and chickpea ingredients. Foods. 2, 338-349 (2013).
16. Gupta, R. K. et al. Health risks and benefits of Chickpea (Cicer arietinum) consumption. J. Agric. Food Chem. 65, 6-22 (2017).
17. Wang, J. et al. Physicochemical Properties of C-Type starch from root tuber of Apios fortunei in comparison with maize, potato, and pea starches. Molecules. 23, 2132 (2018).
18. Paola, R. D. D. Asis, R. & Aldao, M. A. J. Evaluation of the degree of starch gelatinization by a new enzymatic method. Starch. 55, 403-409 (2003).
19. Arnaouteli, S. et al. Pulcherrimin formation controls growth arrest of the Bacillus subtilis biofilm. PNAS. 27, 13553-13562 (2019).
20. Li, X. et al. Identification and high-level production of pulcherrimin in Bacillus licheniformis DW2. Appl Biochem Biotechnol. 183, 1323-1335 (2017).
21. Hadar, K. & Shemesh, M. Probiotic bifunctionality of Bacillus subtilis—Rescuing lactic acid bacteria from desiccation and antagonizing pathogenic Staphylococcus aureus. Microorganisms. 7, 407 (2019).
22. Lahtinen, S. J. Probiotic viability—does it matter? Microb Ecol Health Dis. 23, 10.3402/mehd. v23i0.18567 (2012).
23. Li, W. Novel fermented chickpea milk with enhanced level of γ-aminobutyric acid and neuroprotective effect on PC12 cells. Peer J, 4, e2292 (2016).

### Example 2

### Chickpea Fibers Trigger Pellicle Biofilm Formation by Bacillus subtilis

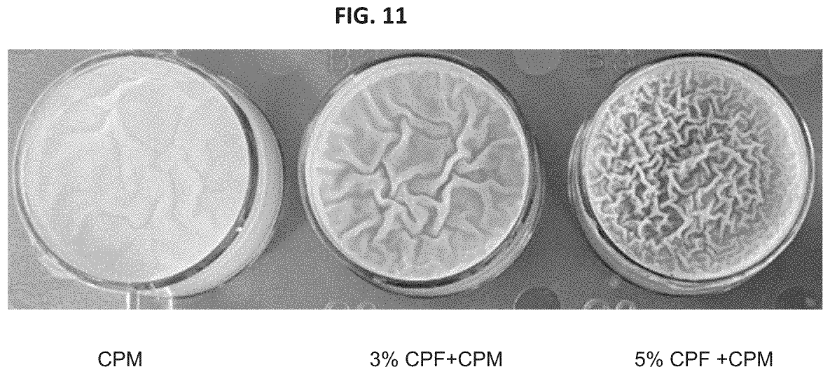

Enrichment of chickpea milk (CPM) with chickpea fiber (CPF) results in improved pellicle formation and secretion of pinkish pigmentation (FIG. 11) associated with pulcherrimin production. Moreover, increasing concentrations of CPF yields induced pellicle and higher pulcherrimin production.

Induction of Biofilm Formation in Regular Laboratory Medium

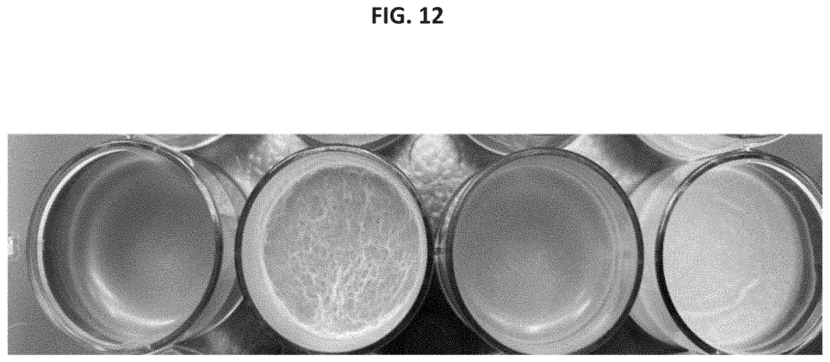

Next, it was determined if CPF has the ability to trigger biofilm formation independently of CPM medium. Lysogeny broth (LB), a laboratory medium that does not support the formation of biofilm pellicles was selected. LB was enriched by different dietary fibers and biofilm formation observed following 48 hours growth of B. subtilis. This was to verify if different types of dietary fibers, such as soluble fibers (wheat fiber) and insoluble fibers (cellulose) were also able to trigger this phenotype. Soluble fibers did not induce biofilm formation in B. subtilis, whereas insoluble fibers triggered the formation of pellicles as shown in FIG. 12.

Growth Rate of B. subtilis in Supplemented Medium

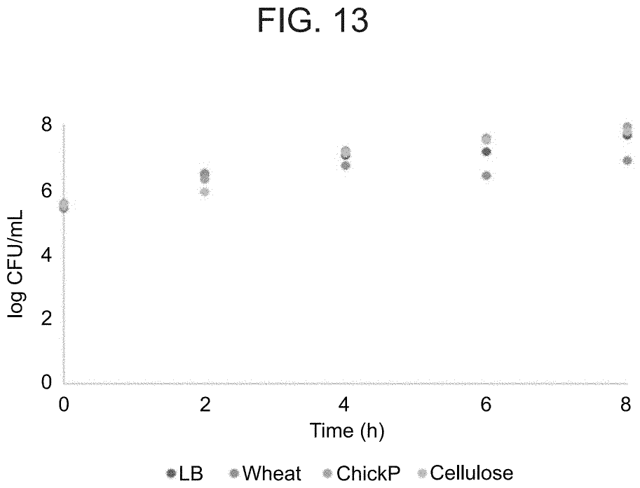

B. subtilis cells grown in the presence of different concentrations of the dietary fibers did not affect the cell growth. Cells grew well in all the different media, with CPF having slightly higher cell counts, as illustrated in FIG. 13.

Survival of B. subtilis Cells During In Vitro Digestion

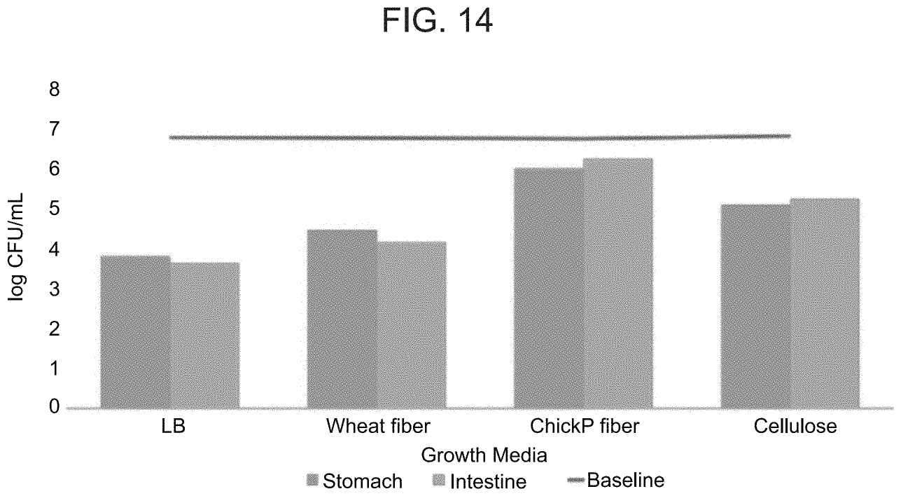

The survivability of B. subtilis grown in the presence of different fibers was compared under simulated gut conditions. The results shown in the FIG. 14 indicate that B. subtilis survived better in the CPF enriched medium compared to all other media. This indicate that the CPF could provide optimum protection for these probiotic cells as they move through the simulated gut.

Confocal Microscopy

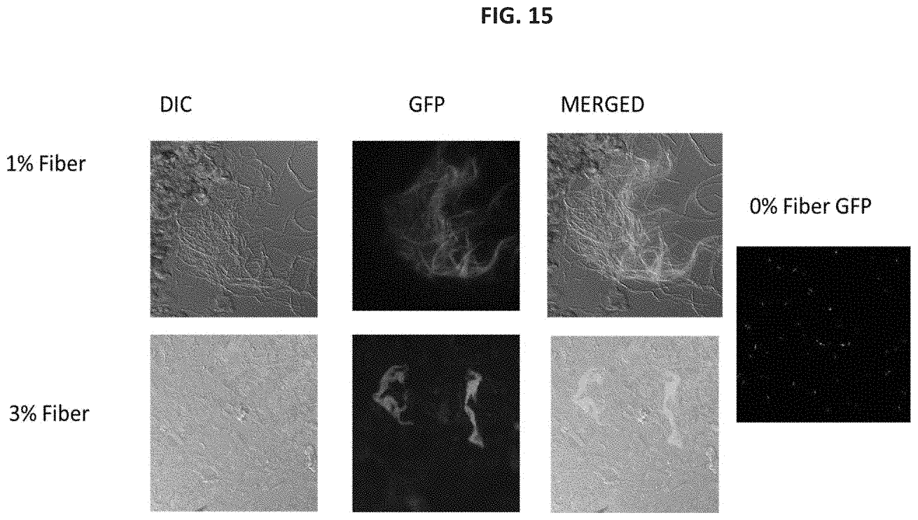

B. subtilis cells grown in CPF showed a general increase in bundling and cell fluorescence with increasing concentrations. This implies that CPF aids in bundling of cells. Increasing concentrations of CPF caused more closely packed cells with more fluorescence, as shown in FIG. 15. From this it may be deduced that higher concentrations of CPF trigger the formation of more bundles, which translates into better and more robust biofilms.

Scanning Electron Microscopy

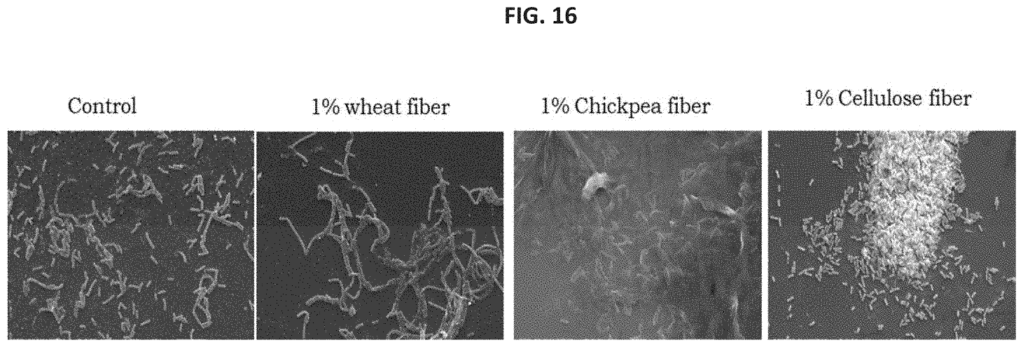

The morphology of B. subtilis was shown to differ under different culturing conditions. In control (LB), cells were observed as separated clusters. In the presence of CPF, the bacterial cells showed significant induction in matrix-embedded bundling (FIG. 16).

Regulation of Matrix Gene Expression in the Presence of Dietary Fibers

A beta-galactosidase assay (Oknin et al., 2015) was used to determine the expression of one of the major matrix operons tapA involved in biofilm formation in the presence of different dietary fibers.

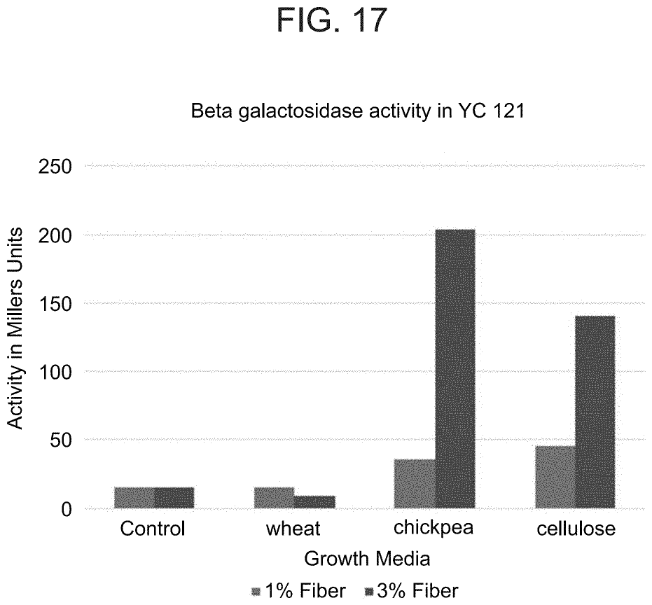

As illustrated in FIG. 17, CPF as well as cellulose fibers significantly induced tapA expression in a dose dependent manner. This implies that insoluble fibers, such as CPF and cellulose trigger biofilm formation via activation of the tapA operon (which is one of the major determinants of biofilm phenotype in B. subtilis).

### REFERENCES

Oknin, H., Steinberg, D., and Shemesh, M. (2015). Magnesium ions mitigate biofilm formation of Bacillus species via downregulation of matrix genes expression. Front Microbiol 6.
Rajasekharan, S. K., Paz-Aviram, T., Galili, S., Berkovich, Z., Reifen, R., and Shemesh, M. (2020). Biofilm formation onto starch fibres by Bacillus subtilis governs its successful adaptation to chickpea milk. Microbial Biotechnology.
Szlufman, C., and Shemesh, M. (2021). Role of Probiotic Bacilli in Developing Synbiotic Food: Challenges and Opportunities. Front Microbiol 12.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of culturing bacteria of the Bacilli class, the method comprising:
(a) adding the bacteria to a medium comprising pasteurized starch fibers of a legume of a leguminous plant, and
(b) culturing said bacteria under conditions that promote generation of a biofilm of said bacteria on said starch fibers, thereby culturing the bacteria.

2. A culture comprising bacteria belonging to the class Bacilli and a medium comprising starch fibers of a pasteurized legume of a leguminous plant.

3. The method of claim 1, wherein said medium is chickpea milk.

4. The culture of claim 2, wherein said medium is chickpea milk.

5. The culture of claim 4, wherein said chickpea milk is fortified with exogenous chickpea starch fibers.

6. The method of claim 3, wherein said chickpea milk is fortified with exogenous chickpea starch fibers.

7. The method of claim 1, wherein said medium is a growth medium selected from the group consisting of lysogeny broth (LB), lysogeny broth enriched with glycerol and manganese (LBGM), milk and Man, Rogosa and Sharpe (MRS) medium.

8. The method of claim 1, wherein said bacteria belong to the genus Bacillus.

9. The method of claim 8, wherein said bacteria are comprised in a biofilm on said starch fibers.

10. The culture of claim 2, wherein said bacteria are comprised in a biofilm on said starch fibers.

11. The method of claim 1, wherein the leguminous plant selected from the group consisting of chickpea (Cicer arietinum), soybean (Glycine max), common bean (Phaseolus vulgaris), pea (Pisum sativum), lentil (Lens culinaris), pigeon pea (Cajanus cajan), broad bean (Vicia faba) and peanut (Arachis hypogaea).

12. The method of claim 11, wherein the leguminous plant is chickpea.

13. The method of claim 1, wherein the bacteria is of a species selected from the group consisting of Bacillus subtilis, Bacillus sonorensis, Bacillus licheniformis, Bacilllus firmus, Bacillus megaterium, B. endophyticus, Bacillus endophyticus and Bacillus amyloliquefaciens.

14. The method of claim 13, wherein the bacteria is of the species Bacillus subtilis.

15. A method of coloring a food product comprising combining a pigment generated by Bacillus subtilis with said food product under conditions that alter the color of the food product, thereby coloring the food product.

16. The method of claim 15, wherein the pigment is comprised in a culture of starch fibers of a legume of a leguminous plant.

17. The method of claim 15, wherein said food product is a meat.

18. The method of claim 15, wherein said pigment is devoid of material of said leguminous plant.

19. A food product comprising the culture of claim 2.

20. The food of claim 19, being a dry snack.
